(19) United States
(12) Patent Application Publication
(10) Pub. No.: US 2025/0352816 A1
(43) Pub. Date: Nov. 20, 2025

(54) DEVICE AND SYSTEM FOR REDUCTION OF TINNITUS DEFECTS AND HEARING DISORDERS IN THE EAR AND TO CORRECT THE DEFECTS ALONG WITH REAL-TIME FEEDBACK WITH THE HELP OF BRAIN OUTPUT SIGNAL RECORDING TECHNOLOGY TO DETERMINE THE AMOUNT OF CORRECTIONS

(71) Applicant: Safa Samadzadeh Etehadi, Tehran (IR)

(72) Inventor: Safa Samadzadeh Etehadi, Tehran (IR)

(21) Appl. No.: 18/668,292

(22) Filed: May 20, 2024

(51) Int. Cl. A61N 2/00; A61N 2/02

(52) U.S. Cl. CPC A61N 2/006; A61N 2/02

(57) ABSTRACT

The invention of a device and system for reduction of tinnitus defects and hearing disorders in the ear and to correct the defects along with real-time feedback with the help of brain output signal recording technology to determine the amount of corrections is related to a device and system to control and reduce hyperacusis, tinnitus and hearing loss and a flexible wearable device that can be placed on a patient's head and also is a portable wearable device that can be connected to a mobile phone and a pair of true wireless earbuds wirelessly. This invention is a system of applying a magnetic field to the auditory cortex of the human brain to reduce hearing defects. This hat is able to simultaneously treat the patient with rTMS and monitor brain activity using fNIRS technology so that it provides accuracy in magnetic stimulation and monitoring of brain reactions.

### TECHNICAL FIELD OF THE INVENTION

The present invention is related to a device and system to control and reduce hyperacusis, tinnitus and hearing loss, and is related to a flexible wearable hat that can be placed on the head of a patient, it is also related to a portable wearable device connecting to a mobile phone wirelessly and related to a pair of true wireless earbuds and also related to a system of applying a magnetic field to the auditory cortex of the human brain to reduce hearing defects.

### PRIOR ART

Ear defects and diseases encompass a variety of conditions that affect the ear and its structure. These conditions can vary based on factors such as gender, genetics, causes, symptoms and outcomes. Infections, acoustic trauma, otologic diseases like Meniere's disease, otosclerosis, presbycusis, immunologic disorder and tumors could cause tinnitus, hearing loss, hyperacusis and other symptoms. Otic inner lining and structural abnormalities, blockage of endolymphatic drainage, trauma, genetic factors and certain medical conditions or drugs tend to these symptoms. Symptoms may be different depending on the effects and may include pain, difficulty in hearing, ringing or buzzing in the ear, excretion, vertigo and imbalance as well as sensitivity to loud noise. Treatment varies depending on underlying causes and severity of conditions from medical treatment and hearing aid to sound therapy, biofeedback and surgery.

### Hyperacusis

Hyperacusis affects 5 to 10% of the population with hearing problems. It is characterized by hypersensitivity to sounds. People with hyperacusis may be hypersensitive to everyday sounds that are normal for everyone else. This disorder can cause serious daily life problems in social and work interactions. Treatment of this disorder usually includes psychological counseling and a combination of different therapies aimed at reducing sensitivity to sounds.

### Tinnitus

Tinnitus occurs widely throughout the world and can range from mild to severe and impactful. An estimated 10 to 20 percent of the population experiences tinnitus, but this number can vary depending on the region and other conditions. This problem leads to the feeling of sounds in the ear without the presence of a sound source. These sounds can include bells, whistles or other sounds. Tinnitus may be stemmed from a variety of problems, including ear injuries, stressors, or certain diseases such as head and neck tumors. Some tinnitus treatments include stress management, the use of noise-blocking devices (such as noise-canceling headphones) and, in some cases, medications.

### Hearing Loss

Hearing loss is one of the common problems in societies. Accurate statistics on its prevalence is highly dependent on factors such as age, genetic, environmental factors, and health conditions. In general, most statistics shows that by aging the population, hearing loss increases. This problem refers to the decrease in hearing power of people. Hearing loss may be stemmed from a variety of factors including age, ear damage, infections, or other diseases. Treatment should be depend on the cause of the hearing loss. Some treatments include hearing aids, surgery, or drug therapy.

The nervous system of the ear is responsible for transmitting hearing information from the ear to the brain and vice versa. This system consists of several different parts, each of which has its own tasks. In general, the nervous system of the ear consists of the following three main parts:

Hearing receptors (Cochlea): Hearing receptors are located in the inner ear and are responsible for converting sound waves into electrical signals. These electrical signals are sent to the brain by auditory neurons.

Auditory Nerve: The auditory nerve or the vestibulocochlear nerve (Cranial Nerve VIII) is responsible for transmitting electrical signals from auditory receptors to the brain. This nerve includes two different parts, each of which transmits hearing and balance information.

Auditory Centers in the Brain: Electrical signals are transmitted from the ear through the auditory nerve to the auditory centers in the brain. These centers are located in different parts of the brain and are responsible for analyzing hearing signals and interpreting them.

In addition to identifying and interpreting sound waves, this complex system is also responsible for maintaining balance in the body. Also, communication with other sensory and motor systems of the body helps to maintain proper balance and coordination in daily activities.

Hyperacusis may be caused by abnormal stimulation or increased sensitivity of nerve cells in the sound processing centers in the brain. This stimulation may be caused by various factors such as injury, inflammation, or chemical changes in the brain. Some studies have shown that disturbances in the central nervous system, including increased activity or changes in the central nervous system, may lead to hyperacusis.

Also, in tinnitus disease, there are fine and tiny hair cells in the inner ear (cochlea) that move by receiving ear sound waves. This movement stimulates electrical signals along the nerve from the ear to the brain (hearing nerve) and the brain interprets these signals as sound. If the hairs of the inner ear are bent or broken due to factors such as very loud noises like explosions, blows to the head or skull, head tumors, ear infections, etc., they can radiate random electrical impulses to the brain, as a result, it causes the stimulation of abnormal activity in the neurons, which leads to the illusion of sound or hearing ringing in the ears.

In the meantime, researchers have presented inventions and methods to treat the defects mentioned in the ear, some of which are mentioned below:

An US invention with patent No. U.S. Ser. No. 10/582,286B2 which is granted dated Mar. 3, 2020 titled “Method for treating debilitating hyperacusis” a system and method of treating hyperacusis is presented. The system uses a customizable, patient-specific, in-ear device combining sound attenuation with loudness suppression and a noise generator to expand the dynamic range of a patient. This device is used with novel software and counseling to provide a patient-specific treatment for hypersensitivity to sound. A method is developed for treating hyperacusis involving a novel transitional device incorporating EPs and sound generators (SGs) and a formal treatment protocol to transition the patient from EPs toward normal audition.

An European invention with patent No. EP1955575B1 which is granted dated 25 Jul. 2012 titled “Apparatus, systems and methods for relieving tinnitus, hyperacusis and/or hearing loss” described a system and method for relieving tinnitus, hyperacusis, and/or hearing loss. One method described includes manipulating an audio signal, associating an audio signal with synchronization information, and transmitting the audio signal and associated synchronization information to a first ear level device and a second ear level device. The method further includes outputting the audio signal substantially simultaneously in the first ear level device and the second ear level device, based at least in part on the synchronization information. The receiver may be capable of communicating with a signal generator to receive signals that are transmitted through the ear level device and thereby heard by a person wearing the ear level device. The signals may comprise audio signals. The signals may be transmitted by radio frequency, WIFI, Bluetooth, and/or similar technologies. The signals may be encoded or otherwise modified to avoid interference with other radio frequencies.

Another US invention with patent No. U.S. Pat. No. 8,979,729B2 which is granted dated 17 Mar. 2015 titled “Tinnitus rehabilitation device and method” described a tinnitus method and device for providing relief to a person suffering from the disturbing effects of tinnitus. The method can be implemented entirely in software to spectrally modify an audio signal in accordance with a predetermined masking algorithm which modifies the intensity of the audio signal at selected frequencies. A predetermined masking algorithm is described which provides intermittent masking of the tinnitus wherein, at a comfortable listening level, during peaks of the audio signal the tinnitus is completely obscured, whereas during troughs the perception of the tinnitus occasionally emerges. In practice, it has been found that such intermittent masking provides an immediate sense of relief, control and relaxation for the person, whilst enabling sufficient perception of the tinnitus for habituation and long term treatment to occur. Advantageously the predetermined masking algorithm is specifically tailored to the audiometric configuration of the person.

In an European invention with patent No. EP2033488B1 which is granted dated 13 Mar. 2013 titled “A hearing aid with an elongate member” type of hearing aid with a housing that is adapted for positioning in the ear canal of a user without obstructing the ear canal and attached to an elongate member that is adapted for positioning in the pinna outside the ear canal of the user is provided. The hearing aid comprises a microphone for converting sound into an audio signal, a signal processor for processing the audio signal into an audio signal compensating a hearing loss, and a loudspeaker that is connected to an output of the signal processor for converting the processed compensated audio signal into a sound signal. Further, the hearing aid comprises a battery for power supply of the electric components of the hearing aid.

Also another US invention with patent No. U.S. Ser. No. 10/356,535B2 which is granted dated 16 Jul. 2019 titled “Method and system for self-managed sound enhancement” provided a system for capturing an individual's hearing characteristics, including loudness tolerance levels at different sound frequencies, as an individualized shaped auditory profile for automatically enhancing audio to complement and address an individual's hearing deficits experienced via a particular signal pathway including the audio device, the sound environment, and the individual's hearing capabilities. The auditory profile is used to produce enhanced sound subsequently transmitted to the individual via the same signal pathway. The enhanced sound may be produced with consideration of both ears' auditory profile shapes together to increase the dynamic range of the enhanced sound. The sound enhancement can be introduced gradually over a sound reproduction session or a period of use.

A Korean invention with patent No. KR101449785B1 which is granted dated 14 Oct. 2014 titled “Auris formulations for treating otic diseases and conditions” discloses compositions and methods for treating ear diseases using immunomodulators and pressure regulators. In this method, the ear compositions and preparations can be applied directly to the middle and/or inner ear target areas with immunomodulators and/or pressure regulator compositions and preparations, or to the middle and/or inner ear structures. A pharmaceutical composition for use in the treatment of ear disease or condition, wherein the composition is administered indoors, on or near a circular window of the ear, Copolymers of polyoxyethylene and polyoxypropylene; and Comprising a multi particulate active agent, wherein the composition provides sustained release of the anti-inflammatory agent for a period of at least 5 days.

Also an US invention with patent No. U.S. Pat. No. 9,913,053B2 which is granted dated Jun. 3, 2018 titled “Sound enrichment for the relief of tinnitus” is a sound enrichment system for the provision of tinnitus relief, the sound enrichment system includes a noise generator, at least one signal modulator for random or pseudo-random modulation of a noise signal that is obtained using the noise generator, and an output transducer for conversion of the modulated noise signal to an acoustic signal for presentation to a user. A method of providing a noise enriched sound signal for provision of relief of tinnitus includes generating a randomly or pseudo-randomly modulated noise signal, generating an acoustic noise signal using the modulated noise signal, and presenting the acoustic noise signal to a tinnitus-suffering person.

Another US invention with patent No. U.S. Ser. No. 10/850,060B2 which is granted dated Jan. 12, 2020 titled “Tinnitus treatment system and method” is a tinnitus masking system for use by a person having tinnitus. The system comprises a sound delivery system having left and right ear-level audio delivery devices and is configured to deliver a masking sound to the person via the audio delivery devices such that the masking sound appears to originate from a virtual sound source location that substantially corresponds to the spatial location in 3D auditory space of the source of the tinnitus as perceived by the person. The masking sound is represented by left and right audio signals that are converted to audible sound by the respective audio delivery devices.

A Chinese invention with patent No. CN111477299B which is granted dated Mar. 1, 2023 titled “Method and device for regulating and controlling sound-electricity stimulation nerves by combining electroencephalogram detection and analysis control” discloses a noninvasive sound and electricity stimulation nerve regulation and control method and device combined with electroencephalogram objective detection analysis feedback control, and the method comprises the following steps: the system comprises an electroencephalogram detection and signal processing and analyzing system, an intelligent diagnosis and treatment control system, an acoustic stimulation treatment system, a transcranial electric pulse stimulation treatment system and a cognitive behavior psychotherapy system, wherein the electroencephalogram detection of a plurality of brain functional areas is carried out on a human body before, during and after noninvasive acoustic stimulation and/or electric pulse stimulation and/or cognitive psychotherapy, and a closed loop of detection, analysis and feedback treatment regulation and control is realized through electroencephalogram signal acquisition, real-time electroencephalogram space-time modeling, objective analysis of characteristic parameters of a neural network and establishment of a correlation model with curative effect evaluation. The method and the device disclosed by the invention can be used for establishing a tracking objective evaluation and prediction method of the multi-course or multi-time nerve regulation and control treatment effect, qualitatively and quantitatively regulating and optimizing a method for implementing a subsequent treatment scheme and selecting scheme parameters according to an expected curative effect, and objectively evaluating and predicting the nerve function state.

And an US invention with publication No. US20200397907A1 which is filed dated Mar. 9, 2020 titled “Triglyceride otic formulations and uses thereof” is a triglyceride based formulations, compositions, and methods for the treatment of otic diseases and conditions. Such triglyceride based formulations and compositions are derived from medium chain triglycerides and allow for the delivery of a variety of therapeutic agents to the outer, middle, and/or inner ear. An otic pharmaceutical formulation comprising: a) a therapeutic agent, or pharmaceutically acceptable prodrug or salt thereof; and b) triglycerides comprising medium chain fatty acids; wherein the triglycerides are present in an amount that is sufficient to stabilize the therapeutic agent for injection into the ear, and wherein the otic pharmaceutical formulation comprises at least about 50% by weight of the triglycerides.

Another US invention with publication No. US20110040205A1 which is filed dated Oct. 11, 2008 titled “Treatment for Alleviating Tinnitus and Hyperacusis with Auditory Stimulation by Compensating for Hearing Loss and Loss of Non-Linear Compressions” is an auditory stimulation treatment for alleviating tinnitus and hyperacusis by compensating for hearing loss and loss of non-linear compression. Natural auditory signals are delivered correcting for hearing loss and compressive non-linearity, both determined at separate frequency bands for the individual subject. This differs from existing treatment in that synthetic “masking” signals are not delivered, but rather the natural auditory input to the subject is modified. Consequently, the method is more akin to a conventional hearing aid. In contrast to conventional hearing aids, however, perception thresholds are specifically corrected and non-linear compression is matched to the specific hearing deficit of the user.

Also an US invention with publication No. US20200228905A1 which is filed dated 31 Mar. 2020 titled “User interface control of multiple parameters for a hearing assistance device” are methods and apparatus for a user interface control to allow control of multiple parameters from a single control for a hearing assistance device. One aspect of the present subject matter relates to hearing assistance device for a wearer, including a housing, hearing assistance electronics housed in the housing, and a tinnitus therapy generator housed in the housing. A user interface control is connected to the electronics and the generator, and the control is configured to sense input from the wearer and provide for selection and adjustment of operational parameters for the electronics and the generator based on the sensed input.

An US invention with publication No. US20140363007A1 which is filed dated 14 Nov. 2006 titled “Wearable device for relieving tinnitus, hyperacusis and/or hearing loss” is provided a wearable device and computerized system and method for configuring a wearable device for use in the treatment of tinnitus, hyperacusis, and/or hearing loss. A software application for use by an audiologist facilitates the diagnosis and assessment of a patient's needs and programming of the wearable device with customized settings and/or audio signals specific to the patient's needs. The customized settings and audio signals may form a part of a tinnitus retraining therapy treatment regime for a patient. The wearable device may further comprise customized settings and sounds for treating hyperacusis and/or hearing loss in a patient. The customized settings can be modified by the audiologist during the fitting process to ensure the patient is fitted with a device that provides appropriate relief for the patient's specific needs.

An European invention with patent No. EP2968010B1 which is granted dated 31 Jan. 2018 “Personalized auditory-somatosensory stimulation to treat tinnitus” relates generally to treatment of tinnitus and, more particularly, to the use of a bimodal stimulation, with stimulation of the auditory and somatosensory systems, to treat tinnitus. Stimulation of both the somatosensory system and the auditory system is achieved, but in a counterintuitive, timed manner, in which spacing between auditory and somatosensory stimuli is used to activate spike-timing dependent plasticity in target neurons in such a manner that spontaneous activity is reduced, thereby reducing or removing tinnitus. Generally, a system for treating an auditory condition in a subject, the system comprising a processor and a memory and a bimodal stimulation system configured to generate an audible stimulation signal having a first firing point and first firing period.

Another European invention with patent No. EP1807154B1 which is granted dated 28 Sep. 2016 titled “Reduction of discomfort using nerve stimulation” relates to the field of transcutaneous stimulation. Specifically, the invention relates to using magnetic fields to reduce discomfort. A system for performing transcutaneous magnetic stimulation, comprising a first magnetic stimulation device for creating a first magnetic field at a first and a second location; and a second magnetic stimulation device for creating a second magnetic field at the second location; characterized in that the second magnetic stimulation device is being adapted to over-stimulate a nerve at the second location with the second magnetic field, wherein the second magnetic stimulation device is being adapted to over-stimulate the nerve at the second location by being pulsed at a rate sufficient to prevent the nerve from repolarizing.

Also an US invention with patent No. U.S. Ser. No. 10/029,111B2 which is granted dated 24 Jul. 2018 titled “rTMS at harmonics of biological signals” repetitive Transcranial Magnetic Stimulation (rTMS) is administered at a pulse rate that is equal to or a harmonic of a biological metric of a patient. The purpose is to provide frequency coupling among different organs (e.g., heart, brain, breathing, and gastrointestinal movement) through rhythmic entrainment. The specific harmonic chosen is the one closest to a desired EEG frequency. The desired EEG frequency is chosen based on the cognitive element or symptom that is targeted. A method of improving a physiological condition or a neuropsychiatric condition of a mammal which comprises subjecting the mammal to repetitive transcranial magnetic stimulation (rTMS) at a frequency of a non-EEG biological metric, or an harmonic or sub-harmonic of said non-EEG biological metric, wherein the mammal is a human.

An US invention with patent No. U.S. Ser. No. 10/350,427B2 which is granted dated 16 Jul. 2019 titled “rTMS device” an rTMS coil or set of coils are designed to affect multiple regions of the brain with synchronous magnetic field pulses. Multiple coils aligned over the targeted regions of interest, or a single coil that is stretched or enlarged in a shape that allows the magnetic field to affect the areas of interest are disclosed. Also disclosed is a method of optimizing repetitive transcranial magnetic stimulation (rTMS) treatments. A repetitive transcranial magnet stimulation (rTMS) device for treating a neuropsychiatric condition or to improve a physiological function, the rTMS device comprises a coil configuration that produces a magnetic field capable of delivering magnetic stimulation, at an rTMS pulse frequency, to two or more regions of a brain of a patient fitted with the rTMS device wherein the rTMS device is configured to analyze recorded signals from the patient and determine, based on the recoded signals, a harmonic or subharmonic of a non-EEG biological metric of the patient.

And An US invention with publication No. US20190201707A1 which is filed dated 28 Dec. 2018 titled “Method and system for therapeutic brain stimulation using electromagnetic pulses” is a therapeutic or diagnostic system comprises a non-invasive brain stimulation device (such as a TMS stimulation device) or other neuromodulation device configured to stimulate a patient's brain or nervous system by emitting electromagnetic pulses according to stimulation parameters, such as a pulse frequency or burst repetition frequency or other parameters, that provides surprising improvements in responsiveness and/or may require only a relatively short train of pulses to achieve high efficacy. In particular, stimulation pulses may be delivered at a frequency of between 12 and 40 Hertz with a 3 to 5 ratio as compared with burst repetition frequency, or at other specific patterns within that range. The stimulation parameters may be pre-stored and customized to individual patients, being identified through an automated search routine during which patient feedback is monitored. A user interface may be provided to allow an operator to conveniently select the appropriate parameters for the desired treatment.

Treatment of these disorders usually involves using a combination of therapies such as psychological counseling, changes in behavior toward sounds, and in some cases medications to reduce sensitivity to sounds.

Specific areas of the brain are targeted by electromagnetic waves in brain stimulation, thereby leads to increasing the number of active cells and improving brain function. It should be noted that stimulation of brain cells have to be applied in a program in the right number and time. In magnetic stimulation of the brain, the coil is placed on the scalp and a point corresponding to the area of the brain that is involved in the creation of the desired neurological symptoms. The coil creates short-term magnetic pulses, and each magnetic pulse easily and painlessly passes through the scalp and bones and layers of the brain and reaches the neurons. It would be called repetitive magnetic stimulation (rTMS) if prescribed consecutively and quickly and is able to create more lasting changes in brain activity. In magnetic stimulation of the brain, a coil is placed above the patient's head so that the person is sitting on a chair and the device connected to it, is placed on the patient's head in the designated area.

rTMS, or Repetitive Magnetic Stimulation, is a non-invasive treatment method used to treat certain psychological and neurological disorders, including hearing disorders such as tinnitus. In this method, a magnetic field is generated that is repeatedly applied to certain areas of the brain. rTMS works in such a way that by producing a strong and variable magnetic field, it stimulates nerves in certain areas of the brain. This stimulation may help to adjust nerve activity in that area of the brain and can have positive effects in the treatment of some neurological and mental disorders.

### DESCRIPTION OF THE INVENTION

The present invention is a device and system equipped with artificial intelligence to reduce, improve and treat ear related diseases, especially hyperacusis, tinnitus and hearing loss. This invention includes two parts, the hat and true wireless earbuds.

The first part of the invention is designed as a flexible and portable hat (FIG. 1, No. 101) and has no location limitations. The hat can be made of polyester, cotton and non-magnetic synthetic fibers that have the ability to transmit the magnetic field and can transmit the magnetic field effectively. Also, the chosen material should be breathable to avoid problems caused by the high heat of the device. A wearable hat is placed on a patient's head (FIG. 2), which is designed in a customized way to fit each patient's head. This hat is designed to cover the entire back of the skull from the forehead to the neck. Also, the ears are free and there is no cover. On both sides of the hat in the temporal lobe section, two coils in a circular shape (FIG. 2 No. 201 and 203) are embedded inside the hat without direct contact with the scalp which are connected to the signal generator and produces electrical signals with a specific frequency and voltage. The signals are applied to the brain with a frequency that corresponds to alpha brain waves (8-12 Hz) and are repeatedly applied to the brain, leading to an increase in the activity of alpha waves in the brain. This magnetic stimulation can lead to an increase the alpha waves activity in the brain, and as a result, improve the individual's state of relaxation and awareness and reduce tinnitus in the patient. The magnetic field in this invention is applied by rTMS method to certain areas of the brain related to the auditory part and affects the neural activity in that area. This magnetic field is applied sequentially and repeatedly to the brain regions. The said magnetic field is sent to the brain through two circular coils and creates an electric current in a specific brain area. The electric current passing through the coil creates a magnetic field that passes through the scalp and skull with little loss and reaches the cortex and significantly improves tinnitus. Also, in the hat of the present invention, fNIRS technology (FIG. 3) is used to receive changes in the shape and frequency of brain waves. In this way, brain waves are simultaneously imaged and the data is sent to the patient's mobile phone via Bluetooth protocol (FIG. 1, No. 102) during the treatment process. Then the data from the mobile phone is sent wirelessly to the artificial intelligence server and is checked, and the results can be viewed and evaluated on the mobile phone.

The said hat is connected to a waist case (FIG. 6) from the back using connecting cable (FIG. 2, No. 204). The connection of hat and case is through a type-C socket (FIG. 7, No. 301) and can be separated from the case when the hat is not needed. Also, half of the connecting cable between the hat and waist case is spring-like so that it has elasticity and does not break the connection between the hat and the case when the patient moves or sits. This case is designed in such a way that it can be easily installed with a handle (FIG. 7 No. 311) in the waist of the patient's clothes. A signal generator (FIG. 7, No. 306), data sending and receiving system (FIG. 7, No. 303), a sound analyzer system for analysis (FIG. 7, No. 307) as well as modifying the frequency and intensity of sounds received from the surroundings, a microphone (FIG. 7 No. 308) to receive sounds from the surrounding environment, a controller (FIG. 7, No. 307) to control the strength and intensity of the magnetic field current produced by the coils, as well as a battery (FIG. 7, No. 312) such as a rechargeable lithium battery are placed inside the case. Based on the results and evaluations of fNIRS imaging, the required settings related to each patient can be entered into the system, so the signal generator can produce electrical signals with the required frequency, intensity and wavelength. The analyzer analyzes the sounds received from the environment and delivers them to the patient's ear with modified intensity and frequency based on the correct hearing range of the patient through True Wireless Earbuds, which will be able to improve hearing loss and hyperacusis to an effective extent.

In the second part, this invention includes a pair of true wireless earbuds (FIG. 8). The surrounding environment sounds are received by the microphone embedded in the waist case and sent to the analyzer section in the case and then analyzed. Corrections are made and the sound is sent to the earbuds with the appropriate intensity and frequency and reaches the patient's ears based on the settings defined for the patient. The aforementioned earbuds have the ability to play music and soothing sounds that help users to reduce their tension and stress during the treatment process. Also, the above-mentioned earbuds can be charged with their own case and can be stored in the case when not in use.

This invention has the ability to adjust the rTMS magnetic stimulation pattern and works precisely to treat hyperacusis, tinnitus or hearing loss with the help of artificial intelligence algorithms. This system has the ability to learn from the person's condition and optimize the treatment. By using the collected data, the artificial intelligence system can automatically optimize the treatment for each person.

Also, the artificial intelligence sensors presented in this invention provide the possibility of monitoring treatment parameters. By using a wireless connection to the mobile device such as Wi-Fi or Bluetooth, the hat enables users to change the settings and control the device easily through the mobile application. Also, the ability to connect the device to a mobile phone makes it possible to measure and monitor data such as brain activity recorded through fNIRS technology and hearing status. This device accurately records information related to each treatment session including treatment progress, brain activity, and hearing status, which can be accessible to users and doctors. Also, this invention allows users to change the settings based on their personal needs through the mobile application and carefully adjust the frequency, intensity and duration of using the device and magnetic stimulation patterns.

In general, the mechanism of action of this invention can include the following steps:

Identification of brain areas related to hearing defects: First, the brain regions related to the desired disease are identified, which can be done through functional hearing evaluations such as tympanometry, acoustic tests, and hearing diagnostic tests. Then the hat and the location of the coils are designed for the desired patient.

Adjusting magnetic stimulation patterns: Magnetic stimulation patterns are adjusted based on the identified brain areas and the data obtained from the fNIRS evaluations. These patterns can stimulate the improvement of electrical currents in the brain areas related to the hearing process and, as a result, help to improve hearing.

Applying magnetic stimulation: After adjusting the stimulation patterns, the rTMS hat applies a magnetic field to the relevant brain areas using special electromagnetic systems. This magnetic stimulation can be sequential and repeated, and their duration and intensity are adjusted based on the hearing needs of each person and the artificial intelligence algorithms of the device.

Receiving and correcting surrounding sounds: If the patient suffers from hearing loss or hyperacusis, the sounds of the surrounding environment are received by the microphone and after correcting and adjusting with the appropriate intensity and frequency, reach the patient's ears through True Wireless Earbuds.

Monitoring and feedback: Information including hearing reactions and brain function during magnetic stimulation is recorded and monitored during treatment sessions. This information can be used for further adjustments and treatment improvement.

Evaluation of progress: In order to evaluate the progress in the treatment, auditory function and brain activity are evaluated simultaneously with the treatment through fNIRS sensors.

As mentioned, the hat should be flexible and soft in order to fit comfortably on the head and allow the wearer to move freely. The magnetic stimulation coils on the hat must be placed in such a way that they can precisely apply the magnetic field to the desired brain areas. These coils must be adjustable. A covering part should be considered that is resistant and waterproof to protect the coils and electronic equipment inside the hat.

Repetitive magnetic stimulation can help to regulate neural activity in the brain's auditory cortex and reduce tinnitus symptoms. The magnetic field applied to the brain area associated with tinnitus is repeated, and specific magnetic stimulation patterns such as intensity and frequency are used, which are directly controlled by the artificial intelligence system.

Also, designing an rTMS hat for the treatment of tinnitus using fNIRS technology can provide an effective improvement in the treatment of this disorder. This hat has the ability for treatment using rTMS simultaneously with monitoring brain activity so that it provides accuracy in magnetic stimulation and monitoring of brain reactions.

### BRIEF DESCRIPTION OF THE FIGURES

FIG. 1 shows an overview of the invention, which includes:
101: Hat
102: Mobile phone
103: waist case
104: True Wireless Earbuds

FIG. 2 shows a view of placing a hat on a human head, which includes:
201: Coil
202: Distribution box for fNIRS sensors wires
203: Coil
204: Cable connecting hat and waist case
205: fNIRS sensor

FIG. 3 shows the fNIRS sensors and coils on the inside of the hat.

FIG. 4 shows how the fNIRS coils and sensors are placed on the brain.

FIG. 5 shows an exploded view of the invention.

FIG. 6 shows the isometric view of the waist case.

FIG. 7 shows the exploded view of the waist case, which includes:
301: type-C cable connecting the hat and waist case
302: Type-C port to connect to the hat
303: sender and receiver
304: front cover of the case
305: Power button
306: signal generator
307: processor consisting of analyzer and controller
308: Microphone
309: charger port
310: Case back cover
311: Waist case holder handle
312: Battery

FIG. 8 shows True Wireless Earbuds and its case.

FIG. 9 shows the manner of placing the hat and waist case on the human body.

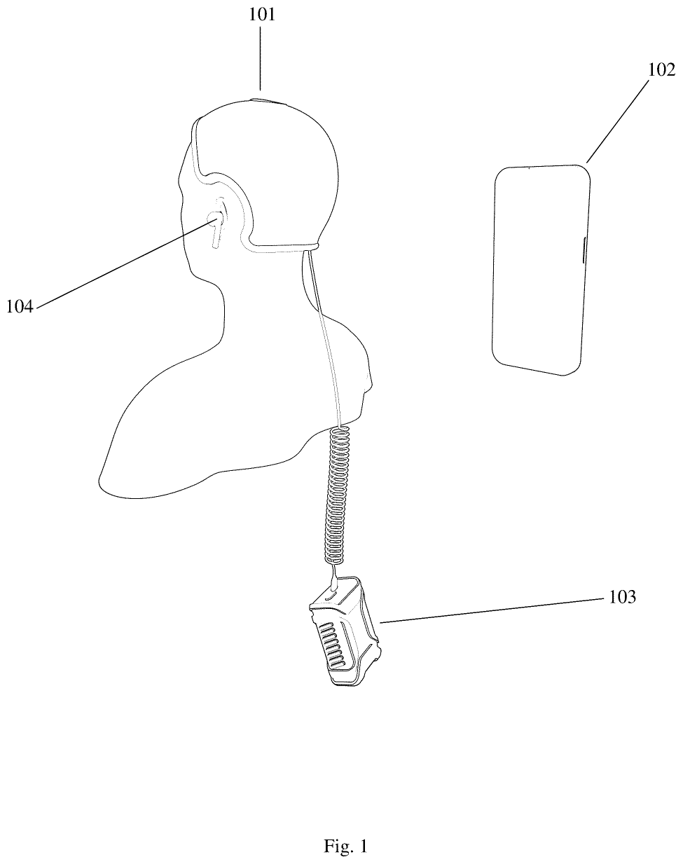

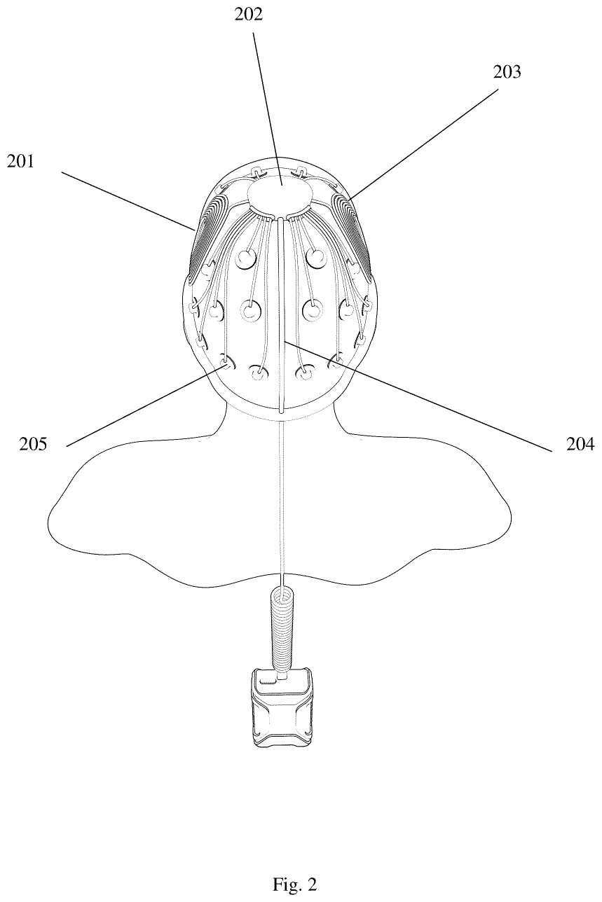

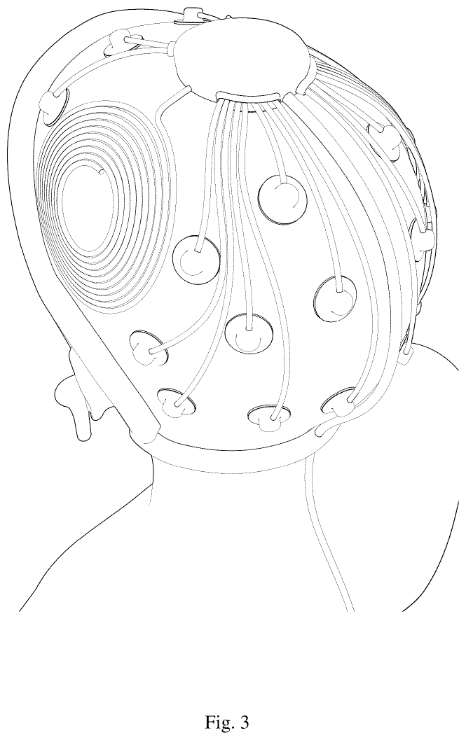

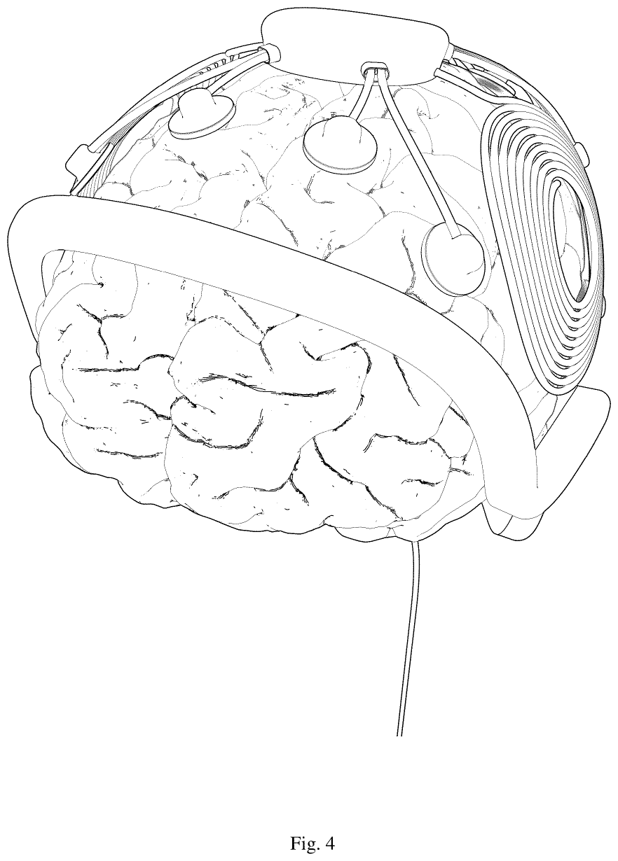

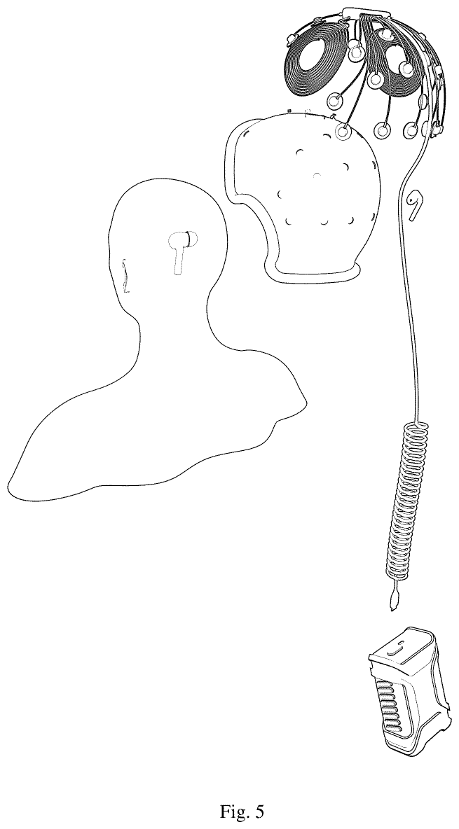

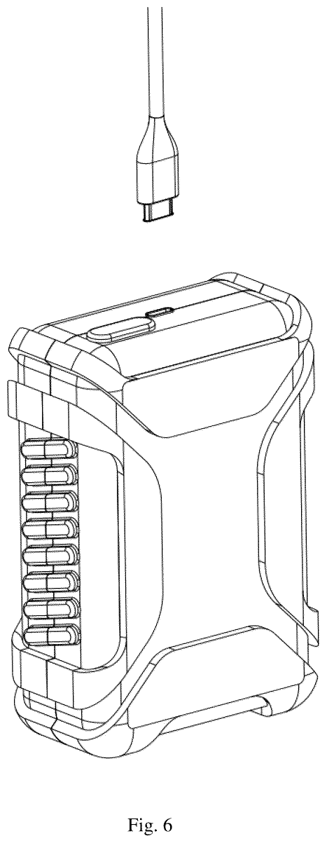

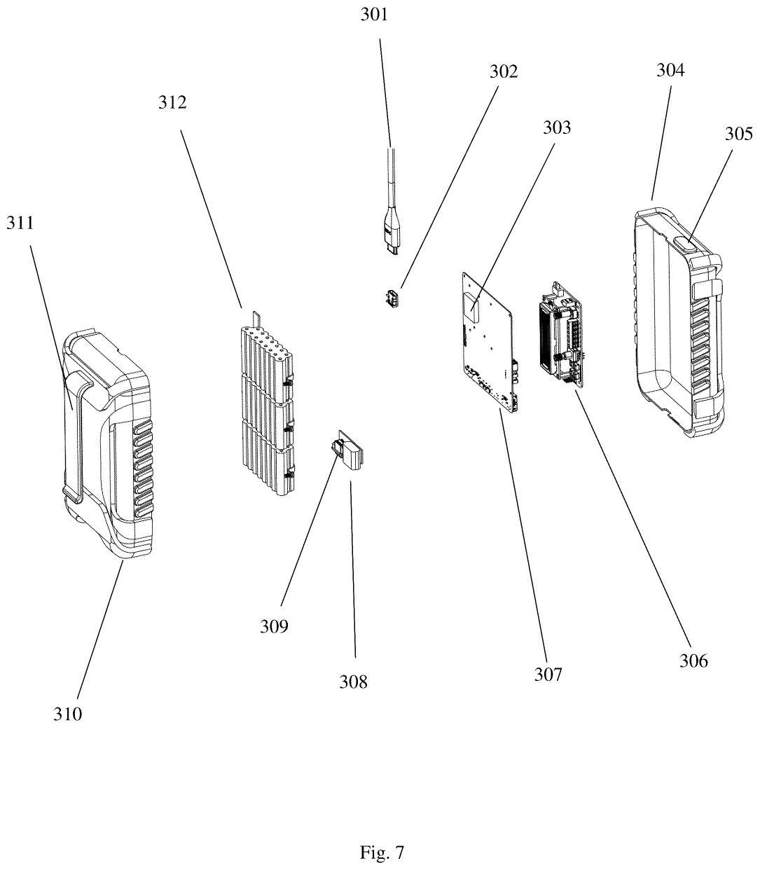

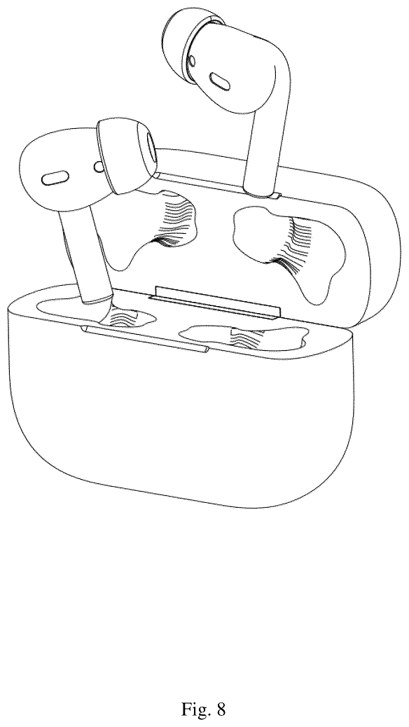

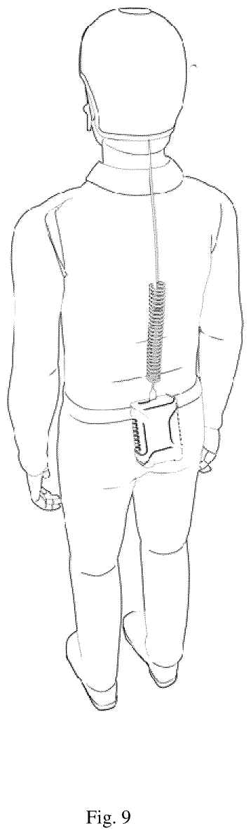

What is claimed is:

1. The invention of a device and system for reduction of tinnitus defects and hearing disorders in the ear and to correct the defects along with real-time feedback with the help of brain output signal recording technology to determine the amount of corrections including at least one flexible hat and at least two coils placed in the hat and at least several fNIRS sensors placed in the hat and at least one pair of True Wireless Earbuds and at least one earbud case and at least one artificial intelligence protocol and at least one mobile application and at least one waist case connected to the hat consisting of at least one signal generator and at least one controller and at least one data sending and receiving system and at least one analyzer and at least one microphone and at least one wireless protocol and at least one battery.

2. The device and system of claim 1 in which there is a portable flexible hat made of polyester, cotton and non-magnetic synthetic fibers, which is designed in a customized way to fit each patient's head.

3. The device and system of claim 1 in which, on both sides of the hat in the temporal lobe section, two coils are embedded in a circular shape inside the hat without direct contact with the scalp.

4. The device and system of claim 1 in which the signals are applied to the brain with a frequency that corresponds to alpha brain waves (8-12 Hz) and are repeatedly applied to the brain, leading to an increase in the activity of alpha waves in the brain.

5. The device and system of claim 1 in which the magnetic field is applied by rTMS method to certain areas of the brain related to the auditory part and affects the neural activity in that area.

6. The device and system of claim 1 in which the magnetic field is sent to the brain through two circular coils and creates an electric current in a specific brain area.

7. The device and system of claim 1 in which fNIRS technology is used to receive changes in the form and frequency of brain waves, and the brain waves are imaged simultaneously with the treatment process and the data is sent to the patient's mobile via Bluetooth protocol.

8. The device and system of claim 1 in which the data from fNIRS imaging is sent wirelessly to the artificial intelligence server from the mobile phone and the results can be viewed and evaluated on the mobile phone.

9. The device and system of claim 1 in which the hat is connected to a waist case with a wire from the back, and the connection between the hat and the case is in the form of a type-C socket, and when the hat is not needed, it can be separated from the case.

10. The device and system of claim 1 in which a microphone is installed on the waist case to receive sounds from the surrounding environment and send to a sound analyzer system to analyze and correct the frequency and intensity of the received sounds.

11. The device and system of claim 1 in which, based on the results and evaluations of fNIRS imaging, the required settings of each patient can be entered into the system so that the signal generator produces electrical signals with the required frequency, intensity and wavelength.

12. The device and system of claim 1 in which there is a pair of true wireless earbuds.

13. The device and system of claim 1 in which, with the help of artificial intelligence algorithms, has the ability to adjust the rTMS magnetic stimulation pattern and it works accurately to treat hyperacusis, tinnitus or hearing loss.

14. The device and system of claim 1 in which artificial intelligence sensors provide the possibility of monitoring parameters related to treatment.

15. The device and system of claim 1 in which the hat, by using a wireless connection such as Wi-Fi or Bluetooth to the mobile device, enables users to change the settings and control the device easily through the mobile application.

16. The device and system of claim 1 in which the ability to connect the device to the mobile makes it possible to measure and monitor data such as brain activity recorded through fNIRS technology and hearing status.
